(19) United States
(12) Patent Application Publication
(10) Pub. No.: US 2025/0152486 A1
(43) Pub. Date: May 15, 2025

(54) Personal Care Compositions and Methods of Using Same

(71) Applicant: Colgate-Palmolive Company, New York, NY (US)

(72) Inventors: Devon Sean COCUZZA, Flemington, NJ (US); Deon HINES, South Bound Brook, NJ (US); Ewelina LESNIAK, North Brunswick, NJ (US); Michael Angelo PEREZ, Roselle Park, NJ (US)

(73) Assignee: Colgate-Palmolive Company, New York, NY (US)

(21) Appl. No.: 18/949,395

(22) Filed: Nov. 15, 2024

Related U.S. Application Data
(60) Provisional application No. 63/599,053, filed on Nov. 15, 2023.

(51) Int. Cl. A61K 8/27; A61K 8/19; A61K 8/35; A61K 8/37; A61K 8/9789; A61Q 17/04

(52) U.S. Cl. CPC A61K 8/27; A61K 8/19; A61K 8/35; A61K 8/37; A61K 8/9789; A61Q 17/04; A61K 2800/30

(57) ABSTRACT

Personal care compositions and, e.g., personal care compositions that achieve enhanced whitecast values for skin types of Fitzpatrick 4, 5, or 6 are disclosed herein. In accordance with an aspect, provided is a personal care composition comprising a mineral UV filter component comprising: a zinc oxide; and an iron oxide system comprising: Fe₃O₄, FeH₃O₃, and Fe₂O₃, wherein the personal care composition has a weight ratio of the FeH₃O₃ to the Fe₃O₄ of about 3.1 to about 6.2 and a weight ratio of the FeH₃O₃ to the Fe₂O₃ of about 2.4 to about 5.1.

### CROSS-REFERENCE TO RELATED APPLICATIONS

This application claims the benefit of priority from U.S. Patent Application No. 63/599,053, filed Nov. 15, 2023, titled PERSONAL CARE COMPOSITIONS AND METHODS OF USING SAME, the content of which is hereby incorporated herein by reference in its entirety for all purposes.

### BACKGROUND

The negative effects of exposure to ultraviolet (“UV”) light are well known. Prolonged exposure to sunlight causes damage such as sunburn to the skin and hair. Exposure to UV light at certain wavelengths, from about 290 nm to about 400 nm, for example, may lead to long-term damage as well as serious conditions such as skin cancer.

For example, ultraviolet radiation in the shorter wavelength UVB region of 290 to 320 nm is considered significant in causing burning. Accordingly, protection against UVB radiation has traditionally been a primary target of sunscreen formulations. Relatively recently there has also been a desire for further protection against UVA radiation to reduce long-term skin deterioration. Generally, UV light contributes to aging by causing free radicals, such as O⁻, OH, superoxide anion, nitric oxide, and hydrogen radicals, to form in the skin. Free radicals attack DNA, membrane lipids, and proteins, generating carbon radicals. These in turn react with oxygen to produce a peroxyl radical that can attack adjacent fatty acids to generate new carbon radicals. The end result is a loss of skin elasticity and the appearance of wrinkles.

More importantly, both the UVA and UVB wavebands have been identified as potential factors for increasing skin cancers, e.g., melanomas. Skin cancer is one of the most common cancer types and the incidence in the US is problematic. For these and other reasons, consistent use of a topical sunscreen is now strongly recommended and is widespread.

Sunscreens are subject to strict regulatory approval by various federal agencies in order to be considered “broad spectrum.” For example, to be considered a “broad spectrum” sunscreen, the current federal standard (21 C.F.R. § 201.327) requires that the mean critical wavelength must be 370 nm or greater. The critical wavelength is identified as the wavelength at which the integral of the spectral absorbance curve reaches 90 percent of the integral over the UV spectrum from 290 to 400 nm. In addition to this requirement, there are regulatory proposals that would require “broad spectrum” sunscreens to meet a UVAI/UV ratio requirement of greater than or equal to 0.70.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention is directed to personal care compositions and particularly personal care compositions that have enhanced whitecast values for skin types of Fitzpatrick 4, 5, or 6. In addition, the inventors discovered that certain preferred embodiments can advantageously achieve enhanced whitecast values for skin types of Fitzpatrick 4, 5, or 6 while simultaneously being broad spectrum sunscreen compositions that additionally provide a UVAI/UV ratio of greater than or equal to 0.70. The personal care compositions disclosed herein may be in the form of a skin care composition, such as a sunscreen composition. In accordance with an aspect, provided is a personal care composition comprising a mineral UV filter component comprising a zinc oxide; and an iron oxide system comprising: Fe₃O₄, FeH₃O₃, and Fe₂O₃, wherein the personal care composition has a weight ratio of the FeH₃O₃ to the Fe₃O₄ of about 3.1 to about 6.2 and a weight ratio of the FeH₃O₃ to the Fe₂O₃ of about 2.4 to about 5.1.

In accordance with another aspect, provided is a personal care composition comprising a mineral UV filter component comprising: a zinc oxide; and an iron oxide system consisting of Fe₃O₄, FeH₃O₃, and Fe₂O₃.

In accordance with a further aspect, provided is a personal care composition comprising a mineral UV filter component comprising from about 9 to about 17 wt. % of a zinc oxide and, optionally, titanium dioxide; and an iron oxide system comprising Fe₃O₄, FeH₃O₃, and Fe₂O₃, wherein the FeH₃O₃ is present in an amount that is greater than the amount of Fe₃O₄ and the FeH₃O₃ is present in an amount that is greater than the amount of Fe₂O₃, wherein all weight percentages are based on the total weight of the personal care composition.

In some embodiments, the term “wt. %”, and the like, refers to weight percentage based on the total weight of the personal care composition

A list of non-limiting example embodiments is provided below:

In an embodiment 1, provided is a personal care composition (e.g., preferably a sunscreen composition, more preferably a broad spectrum sunscreen composition) comprising: a mineral UV filter component comprising: a zinc oxide; and an iron oxide system comprising: Fe₃O₄, FeH₃O₃, and Fe₂O₃; wherein the personal care composition has a weight ratio of the FeH₃O₃ to the Fe₃O₄ of about 3.1 to about 6.2 and a weight ratio of the FeH₃O₃ to the Fe₂O₃ of about 2.4 to about 5.1, and preferably wherein the personal care composition has a SPF value of about 40 or more and a UVAI/UV ratio of preferably about 72 or more.

In an embodiment 2, provided is a personal care composition according to embodiment 1, wherein the iron oxide system is present in an amount from about 0.6 to about 2.1 wt. %, based on the total weight of the personal care composition.

In an embodiment 3, provided is a personal care composition according to embodiment 1 or embodiment 2, wherein the iron oxide system is present in an amount from about 0.8 to about 1.25 wt. %, optionally about 0.9 to about 1 wt. %, based on the total weight of the personal care composition.

In an embodiment 4, provided is a personal care composition according to embodiment 1 or embodiment 2, wherein the iron oxide system is present in an amount from about 1.25 to about 2.1 wt. %, optionally about 1.4 to about 1.9 wt. %, based on the total weight of the personal care composition.

In an embodiment 5, provided is a personal care composition according to any foregoing embodiment, wherein the weight ratio of the FeH₃O₃ to the Fe₃O₄ of about 3.2 to about 6, optionally about 3.8 to about 5.1.

In an embodiment 6, provided is a personal care composition according to any foregoing embodiment, wherein the weight ratio of the FeH₃O₃ to the Fe₂O₃ of about 2.6 to about 4.9, optionally about 3.1 to about 4.2.

In an embodiment 7, provided is a personal care composition according to any foregoing embodiment, wherein the mineral UV filter is present in the amount of about 5 to about 14 wt. %, optionally about 6 to about 12 wt. %, based on the total weight of the personal care composition.

In an embodiment 8, provided is a personal care composition according to any foregoing embodiment further comprising an organic UV filter, optionally present in an amount from about 1 to about 18 wt. %, based on the total weight of the personal care composition.

In an embodiment 9, provided is a personal care composition according to embodiment 8, wherein the organic UV filter is selected from ethylhexyl methoxycinnamate, oxybenzone, homosalate, octisalate, octocrylene, and a combination of two or more thereof.

In an embodiment 10, provided is a personal care composition according to any foregoing embodiment further comprising a fatty compound, optionally, in an amount about 1 to about 10 wt. %, based on the total weight of the personal care composition.

In an embodiment 11, provided is a personal care composition according to any foregoing embodiment further comprising a silicone oil, optionally, in an amount about 1 to about 10 wt. %, based on the total weight of the personal care composition.

In an embodiment 12, provided is a personal care composition according to any foregoing embodiment further comprising: a SPF enhancer comprising butyloctyl salicylate, ethylhexyl methoxycrylene, octyldodecyl neopentanoate, Pongamia pinnata extract, or a combination of two or more thereof.

In an embodiment 13, provided is a personal care composition according to any foregoing embodiment, wherein the amino acid comprises a plurality of amino acids selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

In an embodiment 14, provided is a personal care composition according to any foregoing embodiment, wherein the plurality of amino acids is selected from glycine, arginine, taurine, histidine, tyrosine, tryptophan, a salt thereof, and a combination of two or more thereof.

In an embodiment 15, provided is a personal care composition according to any foregoing embodiment, wherein the personal care composition is free of titanium dioxide.

In an embodiment 16, provided is a personal care composition according to any of embodiment 1 to embodiment 15, further comprising titanium dioxide, optionally in an amount from about 0.4 to about 8 wt. %, based on the total weight of the personal care composition.

In accordance with an embodiment 17, provided is a personal care composition (e.g., preferably a sunscreen composition, more preferably a broad spectrum sunscreen composition) comprising: a mineral UV filter component comprising: a zinc oxide; and an iron oxide system consisting of Fe₃O₄, FeH₃O₃, and Fe₂O₃, and preferably wherein the personal care composition has a SPF value of about 40 or more and a UVAI/UV ratio of preferably about 72 or more.

In an embodiment 18, provided is a personal care composition according to embodiment 17, wherein the FeH₃O₃ is present in an amount that is greater than the amount of Fe₃O₄.

In an embodiment 19, provided is a personal care composition according to embodiment 17 or embodiment 18, wherein the FeH₃O₃ is present in an amount that is greater than the amount of Fe₂O₃.

In an embodiment 20, provided is a personal care composition according to one of embodiment 17 to embodiment 19 further comprising an organic UV filter selected from ethylhexyl methoxycinnamate, oxybenzone, homosalate, octisalate, octocrylene, and a combination of two or more thereof, and preferably wherein the personal care composition has a SPF value of about 40 or more and a UVAI/UV ratio of preferably about 72 or more.

In an embodiment 21, provided is a personal care composition (e.g., preferably a sunscreen composition, more preferably a broad spectrum sunscreen composition) comprising: a mineral UV filter component comprising: from about 9 to about 17 wt. % of a zinc oxide; and optionally, titanium dioxide; and an iron oxide system comprising: Fe₃O₄, FeH₃O₃, and Fe₂O₃, wherein the FeH₃O₃ is present in an amount that is greater than the amount of Fe₃O₄ and the FeH₃O₃ is present in an amount that is greater than the amount of Fe₂O₃.

In an embodiment 22, provided is a personal care composition according to embodiment 21 comprising titanium dioxide, wherein the titanium dioxide is present in an amount from about 0.4 to about 8 wt. %, based on the total weight of the personal care composition.

In an embodiment 23, provided is a personal care composition according to embodiment 21 or embodiment 22, wherein the iron oxide system is present in an amount from about 1 to 7 wt. %, based on the total weight of the personal care composition.

In an embodiment 24, provided is a personal care composition according to one of embodiment 21 to embodiment 22 further comprising an organic UV filter, optionally present in an amount from about 1 to about 18 wt. %, based on the total weight of the personal care composition.

In an embodiment 25, provided is a personal care composition according to 24, wherein the organic UV filter is selected from ethylhexyl methoxycinnamate, oxybenzone, homosalate, octisalate, octocrylene, and a combination of two or more thereof.

In an embodiment 26, provided is a personal care composition according to one of embodiment 21 to embodiment 25, wherein the personal care composition has a weight ratio of the FeH₃O₃ to the Fe₃O₄ of about 3.1 to about 6.2, preferably from about 4.2:1 to about 5.5:1.

In an embodiment 27, provided is a personal care composition according to one of embodiment 21 to embodiment 26, wherein the personal care composition has a weight ratio of the FeH₃O₃ to the Fe₂O₃ of about 2.4 to about 5.1, preferably about 3.4:1 to about 4.2.

In an embodiment 28, provided is a personal care composition (e.g., preferably a sunscreen composition, more preferably a broad spectrum sunscreen composition) comprising: a mineral UV filter component comprising: a zinc oxide; and an iron oxide system comprising: iron (II,III) oxide, yellow iron oxide, and red ferric oxide; wherein the personal care composition has a weight ratio of the yellow iron oxide to the iron (II,III) oxide of about 3.1 to about 6.2 and a weight ratio of the yellow iron oxide to the red ferric oxide of about 2.4 to about 5.1, and preferably wherein the personal care composition has a SPF value of about 40 or more and a UVAI/UV ratio of preferably about 72 or more.

In accordance with an embodiment 29, provided is a use of an iron oxide system comprising iron (II,III) oxide, yellow iron oxide, and red ferric oxide to reduce whitecast, enhance SPF value, and/or enhance UVAI/UV ratio in a sunscreen composition (e.g., preferably a broad spectrum sunscreen composition) having a zinc oxide, a SPF value of about 40 or more and a UVAI/UV ratio of preferably about 72 or more.

In accordance with an embodiment 30, provided is a use of an iron oxide system comprising iron (II,III) oxide, yellow iron oxide, and red ferric oxide to reduce whitecast in a sunscreen composition (e.g., preferably a broad spectrum sunscreen composition) having a zinc oxide, a SPF value of about 40 or more and a UVAI/UV ratio of preferably about 72 or more.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “hr”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

“Volatile”, as used herein, means having a flash point of less than about 100° C. “Non-volatile”, as used herein, means having a flash point of greater than about 100° C.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

Any member in a list of species that is used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that defines or exemplifies the genus is open, and it is given that other species may exist that define or exemplify the genus just as well as, or better than, any other species listed.

The phrases, “a mixture thereof,” “a combination thereof,” or “a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a humectant and a polyol. If a particular personal care composition includes and/or recites both a humectant and a polyol, a compound that may be characterized as both a humectant and a polyol, such as glycerin, will serve only as either the humectant or the polyol—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to personal care compositions and, e.g., personal care compositions that have improved whitecast values for skin types of Fitzpatrick 4, 5, or 6. The personal care compositions may be in the form of a skin care composition, such as a sunscreen composition.

It typically understood that it is difficult to develop personal care compositions, such as sunscreen compositions, having ZnO and or TiO2, which are high refractive index materials and as a result tend to appear white on the skin (whitecast). Personal care compositions resulting in a whitecast on skin are less cosmetically elegant and often less desirable to consumers. Additionally, attempts to reduce whitecast often result in the composition making the skin appear unnatural dark.

The inventors discovered that personal care compositions having particular mineral UV filter components comprising certain iron oxide systems can unexpectedly provide preferable SPF values and UVAI/UV ratios while simultaneously achieving desirable whitecast values for skin types of Fitzpatrick 4, 5, or 6. For instance, certain embodiments disclosed herein overcome many of the difficulties of formulating personal care compositions, such as sunscreen compositions, having mineral UV filter components that provide both desirable SPF values (e.g., SPF values of preferably about 44 or more, preferably about 47 or more, preferably about 49 or more, preferably about 51 or more, preferably about 53 or more, preferably about 56 or more, preferably about 60 or more, preferably about 62 or more, preferably about 64 or more, preferably about 65 or more, preferably about 67 or more, preferably about 69 or more, preferably about 70 or more, preferably about 72 or more, preferably about 75 or more, preferably about 78 or more, or preferably about 80 or more) and UVAI/UV ratios (e.g., UVAI/UV ratio of preferably about 72 or more, preferably about 74 or more, preferably about 76 or more, preferably about 78 or more, preferably about 80 or more, or preferably about 82 or more) while reducing and/or eliminating undesirable whitecast values for skin types of Fitzpatrick 4, 5, or 6.

In accordance with an aspect, provided is a personal care composition comprising a mineral UV filter component comprising: a zinc oxide; and an iron oxide system comprising: Fe₃O₄, FeH₃O₃, and Fe₂O₃, wherein the personal care composition has a weight ratio of the FeH₃O₃ to the Fe₃O₄ of about 3.1 to about 6.2 and a weight ratio of the FeH₃O₃ to the Fe₂O₃ of about 2.4 to about 5.1. The personal care composition may, preferably, have a SPF value of 40 or more and a UVAI/UV ratio greater than or equal to 70.

In accordance with another aspect, provided is a personal care composition comprising a mineral UV filter component comprising: a zinc oxide; and an iron oxide system consisting of Fe₃O₄, FeH₃O₃, and Fe₂O₃. The personal care composition may, preferably, have a SPF value of 40 or more and a UVAI/UV ratio greater than or equal to 70.

In accordance with a further aspect, provided is a personal care composition comprising a mineral UV filter component comprising from about 9 to about 17 wt. % of a zinc oxide and, optionally, titanium dioxide; and an iron oxide system comprising Fe₃O₄, FeH₃O₃, and Fe₂O₃, wherein the FeH₃O₃ is present in an amount that is greater than the amount of Fe₃O₄ and the FeH₃O₃ is present in an amount that is greater than the amount of Fe₂O₃, wherein all weight percentages are based on the total weight of the personal care composition. The personal care composition may, preferably, have a SPF value of 40 or more and a UVAI/UV ratio greater than or equal to 70.

The personal care compositions may achieve a SPF value of 40 or more in some embodiments. For instance, the personal care composition may have a SPF value of about 44 or more, about 47 or more, about 49 or more, about 51 or more, about 53 or more, about 56 or more, about 60 or more, about 62 or more, about 64 or more, about 65 or more, about 67 or more, about 69 or more, about 70 or more, about 72 or more, about 75 or more, about 78 or more, or about 80 or more. Additionally or alternatively, the personal care composition may have a UVAI/UV ratio greater than or equal to 70. In certain embodiments, the personal care composition has a UVAI/UV ratio of about 72 or more, about 74 or more, about 76 or more, about 78 or more, about 80 or more, or about 82 or more.

In some embodiments, the personal care compositions is formulated to have a reduced amount, be substantially free of, or be free of titanium dioxide. For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of titanium dioxide, based on the total weight of the personal care composition. In at least one embodiment, the personal care composition contains about 0 wt. % or 0 wt. % of titanium dioxide, based on the total weight of the personal care composition.

Additionally or alternatively, the personal care compositions may be formulated to have a reduced amount, be substantially free of, or be free of titanium compounds. For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of titanium compounds, based on the total weight of the personal care composition. In at least one embodiment, the personal care composition contains about 0 wt. % or 0 wt. % of titanium compounds, based on the total weight of the personal care composition.

Suitable components, such as those listed below, may be included or excluded from the formulations for the personal care compositions depending on the specific combination of other ingredients, the form of the personal care composition, and/or the use of such composition. For example, the personal care composition may include a hydrophilic phase and a hydrophobic phase when the personal care composition is in the form of an oil-in-water emulsion, a water-in-oil emulsion, or a two-phase composition having a first phase, a second, phase, and a single interphase therebetween. In some embodiments, the personal care composition is in the form of a skin care composition, such as a sunscreen composition.

The personal care composition typically comprises one or more mineral UV filter(s), preferably in an effective amount. In some instances, the amount of mineral UV filter(s) in the personal care composition is from 5 to about 25 wt. %, based on the total weight of the personal care composition. For example, the mineral UV filter(s) may be present in the personal care composition in an amount from about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 5 to about 18 wt. %, about 7 to about 18 wt. %, about 9 to about 15 wt. %, about 11 to about 18 wt. %, about 13 to about 18 wt. %, about 15 to about 18 wt. %; from about 5 to about 15 wt. %, about 7 to about 15 wt. %, about 9 to about 15 wt. %, about 11 to about 15 wt. %, about 13 to about 15 wt. %; from about 5 to about 13 wt. %, about 7 to about 13 wt. %, about 9 to about 13 wt. %, about 11 to about 13 wt. %; from about 5 to about 11 wt. %, about 7 to about 11 wt. %, about 9 to about 11 wt. %; from about 5 to about 9 wt. %, about 7 to about 9 wt. %; from about 5 to about 8 wt. %, about 7 to about 8 wt. %, about 5 to about 7 wt. %, or any range or subrange thereof, based on the total weight of the personal care composition.

The mineral UV filters may comprise metal oxides, including titanium oxide (amorphous or crystallized in rutile and/or anatase form), iron oxide, zinc oxide, zirconium oxide, and/or cerium oxide. The metal oxides may comprise treated or untreated metal oxides. In some embodiments, the mineral UV filter is selected from iron oxide, zinc oxide, zirconium oxide, cerium oxide, and a combination thereof. For example, the mineral UV filter may include, or in some cases consist of, one or more iron oxide, zinc oxide, zirconium oxide, or a combination of two or more thereof. In at least one preferred embodiment, the mineral UV filters are selected from the group consisting of one or more iron oxide, zinc oxide, zirconium oxide, and a combination of two or more thereof. In at least one embodiment, the personal care composition includes one or more zinc oxide(s). The personal care composition, in some instances, may consist of mineral UV filters selected from zinc oxides.

The personal care compositions typically comprise an iron oxide system. The iron oxide system may be present in an amount from about 0.6 to about 2.1 wt. %, based on the total weight of the personal care composition. For instance, in some embodiments, the iron oxide system is present in an amount from about 0.8 to about 1.25 wt. %, about 0.8 to about 1.2 wt. %, about 0.8 to about 1.15 wt. %, about 0.8 to about 1.1 wt. %, about 0.8 to about 1.05 wt. %, about 0.8 to about 1 wt. %, about 0.8 to about 0.95 wt. %, about 0.8 to about 0.9 wt. %; from about 0.85 to about 1.25 wt. %, about 0.85 to about 1.2 wt. %, about 0.85 to about 1.15 wt. %, about 0.85 to about 1.1 wt. %, about 0.85 to about 1.05 wt. %, about 0.85 to about 1 wt. %, about 0.85 to about 0.95 wt. %; from about 0.9 to about 1.25 wt. %, about 0.9 to about 1.2 wt. %, about 0.9 to about 1.15 wt. %, about 0.9 to about 1.1 wt. %, about 0.9 to about 1.05 wt. %, about 0.9 to about 1 wt. %; from about 0.95 to about 1.25 wt. %, about 0.95 to about 1.2 wt. %, about 0.95 to about 1.15 wt. %, about 0.95 to about 1.1 wt. %, about 0.95 to about 1.05 wt. %; from about 1 to about 1.25 wt. %, about 1 to about 1.2 wt. %, about 1 to about 1.15 wt. %, about 1 to about 1.1 wt. %, or any range or subrange thereof, based on the total weight of the personal care composition.

In some embodiments, the iron oxide system is present in the personal care composition in an amount from about 1.25 to about 2.1 wt. %, based on the total weight of the personal care composition. For example, the personal care composition may comprise the iron oxide system in an amount from about 1.25 to about 2.1 wt. %, about 1.25 to about 2 wt. %, about 1.25 to about 1.9 wt. %, about 1.25 to about 1.8 wt. %, about 1.25 to about 1.7 wt. %, about 1.25 to about 1.6 wt. %, about 1.25 to about 1.5 wt. %; from about 1.3 to about 2.1 wt. %, about 1.3 to about 2 wt. %, about 1.3 to about 1.9 wt. %, about 1.3 to about 1.8 wt. %, about 1.3 to about 1.7 wt. %, about 1.3 to about 1.6 wt. %, about 1.3 to about 1.5 wt. %; from about 1.4 to about 2.1 wt. %, about 1.4 to about 2 wt. %, about 1.4 to about 1.9 wt. %, about 1.4 to about 1.8 wt. %, about 1.4 to about 1.7 wt. %, about 1.4 to about 1.6 wt. %; from about 1.5 to about 2.1 wt. %, about 1.5 to about 2 wt. %, about 1.5 to about 1.9 wt. %, about 1.5 to about 1.8 wt. %, about 1.5 to about 1.7 wt. %; from about 1.6 to about 2.1 wt. %, about 1.6 to about 2 wt. %, about 1.6 to about 1.9 wt. %, about 1.6 to about 1.8 wt. %; from about 1.7 to about 2.1 wt. %, about 1.7 to about 2 wt. %, about 1.7 to about 1.9 wt. %; from about 1.8 to about 2.1 wt. %, about 1.8 to about 2 wt. %; from about 1.9 to about 2.1 wt. %, about 1.9 to about 2 wt. %, or any range or subrange thereof, based on the total weight of the personal care composition.

The iron oxide system typically comprises a plurality of iron oxide. The iron oxide system may comprise iron (II,III) oxide, yellow ferric oxide, red ferric oxide, or a combination thereof. Preferably, the personal care composition comprises at least two of iron (II,III) oxide, yellow ferric oxide, or red ferric oxide. For instance, the personal care composition may include yellow ferric oxide and red ferric oxide. Additionally or alternatively, the personal care composition may include yellow ferric oxide and iron (II,III) oxide. In some embodiments, the personal care composition comprises iron (II,III) oxide, yellow ferric oxide, and red ferric oxide.

The yellow ferric oxide may comprise FeH₃O₃, FeOH₃, or a combination thereof. In some embodiments, the yellow ferric oxide consists of at least one of FeH₃O₃ and FeOH₃, and optionally impurities. The red ferric oxide may comprise Fe₂O₃. In at least one embodiment, the red ferric oxide consists of Fe₂O₃ and optionally impurities. The iron (II,III) oxide may comprise Fe₃O₄. In some embodiments, the iron (II,III) oxide consists of Fe₃O₄ and optionally impurities.

Additionally or alternatively, the iron oxide component may comprise FeH₃O₃, Fe₂O₃, Fe₃O₄, or a combination thereof. For example, the iron oxide system may comprise at least two of FeH₃O₃, Fe₂O₃, or Fe₃O₄. In some embodiments, the iron oxide system comprises FeH₃O₃ and Fe₂O₃. In further embodiments, the iron oxide system comprises FeH₃O₃ and Fe₃O₄. In at least one embodiment, the iron oxide system comprises FeH₃O₃, Fe₂O₃, and Fe₃O₄. The iron oxide system may, in some preferrable embodiments, consist of FeH₃O₃, Fe₂O₃, Fe₃O₄, and optionally impurities.

Without being limited to any particular theory, it is believed that personal care compositions having particular iron oxide systems with certain weight ratios significantly improve the whitecast value on skin types of Fitzpatrick 4, 5, or 6. In certain embodiments, the personal care composition have a weight ratio of FeH₃O₃ and/or yellow oxide to the Fe₃O₄ and/or iron (II,III) oxide of about 3.1 to about 6.2. For instance, the personal care composition may preferably have a weight ratio of FeH₃O₃ and/or yellow oxide to the Fe₃O₄ and/or iron (II,III) oxide of about 3.1 to about 5.9, about 3.1 to about 5.7, about 3.1 to about 5.5, about 3.1 to about 5.3, about 3.1 to about 5.1, about 3.1 to about 4.9, about 3.1 to about 4.7, about 3.1 to about 4.5, about 3.1 to about 4.3, about 3.1 to about 4.1; from about 3.3 to about 6.2, about 3.3 to about 5.9, about 3.3 to about 5.7, about 3.3 to about 5.5, about 3.3 to about 5.3, about 3.3 to about 5.1, about 3.3 to about 4.9, about 3.3 to about 4.7, about 3.3 to about 4.5, about 3.3 to about 4.3, about 3.3 to about 4.1; from about 3.6 to about 6.2, about 3.6 to about 5.9, about 3.6 to about 5.7, about 3.6 to about 5.5, about 3.6 to about 5.3, about 3.6 to about 5.1, about 3.6 to about 4.9, about 3.6 to about 4.7, about 3.6 to about 4.5, about 3.6 to about 4.3; from about 3.8 to about 6.2, about 3.8 to about 5.9, about 3.8 to about 5.7, about 3.8 to about 5.5, about 3.8 to about 5.3, about 3.8 to about 5.1, about 3.8 to about 4.9, about 3.8 to about 4.7, about 3.8 to about 4.5, about 3.8 to about 4.3; from about 4 to about 6.2, about 4 to about 5.9, about 4 to about 5.7, about 4 to about 5.5, about 4 to about 5.3, about 4 to about 5.1, about 4 to about 4.9, about 4 to about 4.7, about 4 to about 4.5; from about 4.2 to about 6.2, about 4.2 to about 5.9, about 4.2 to about 5.7, about 4.2 to about 5.5, about 4.2 to about 5.3, about 4.2 to about 5.1, about 4.2 to about 4.9, about 4.2 to about 4.7; from about 4.4 to about 6.2, about 4.4 to about 5.9, about 4.4 to about 5.7, about 4.4 to about 5.5, about 4.4 to about 5.3, about 4.4 to about 5.1, about 4.4 to about 4.9; from about 4.6 to about 6.2, about 4.6 to about 5.9, about 4.6 to about 5.7, about 4.6 to about 5.5, about 4.6 to about 5.3, about 4.6 to about 5.1, or any range or subrange thereof. In at least one preferred embodiment, the personal care composition has a weight ratio of the FeH₃O₃ and/or yellow oxide to the Fe₃O₄ and/or iron (II,III) of about 3.1 to about 6.2, preferably from about 4.2:1 to about 5.5:1.

Additionally or alternatively, the personal care composition may have a preferable weight ratio of the FeH₃O₃ and/or yellow iron oxide to the Fe₂O₃ and/or red iron oxide of about 2.4 to about 5.1. In some preferred embodiments, the personal care composition has a weight ratio of the FeH₃O₃ to the Fe₂O₃ of about 2.4 to about 5.1, about 2.4 to about 4.8, about 2.4 to about 4.6, about 2.4 to about 4.4, about 2.4 to about 4.2, about 2.4 to about 4, about 2.4 to about 3.8, about 2.4 to about 3.6, about 2.4 to about 3.4; from about 2.7 to about 5.1, about 2.7 to about 4.8, about 2.7 to about 4.6, about 2.7 to about 4.4, about 2.7 to about 4.2, about 2.7 to about 4, about 2.7 to about 3.8, about 2.7 to about 3.6, about 2.7 to about 3.4; from about 2.9 to about 5.1, about 2.9 to about 4.8, about 2.9 to about 4.6, about 2.9 to about 4.4, about 2.9 to about 4.2, about 2.9 to about 4, about 2.9 to about 3.8, about 2.9 to about 3.6, about 2.9 to about 3.4; from about 3.1 to about 5.1, about 3.1 to about 4.8, about 3.1 to about 4.6, about 3.1 to about 4.4, about 3.1 to about 4.2, about 3.1 to about 4, about 3.1 to about 3.8; from about 3.3 to about 5.1, about 3.3 to about 4.8, about 3.3 to about 4.6, about 3.3 to about 4.4, about 3.3 to about 4.2, about 3.3 to about 4, about 3.3 to about 3.8; from about 3.5 to about 5.1, about 3.5 to about 4.8, about 3.5 to about 4.6, about 3.5 to about 4.4, about 3.5 to about 4.2, about 3.5 to about 4, about 3.5 to about 3.8; from about 3.7 to about 5.1, about 3.7 to about 4.8, about 3.7 to about 4.6, about 3.7 to about 4.4, about 3.7 to about 4.2; from about 3.9 to about 5.1, about 3.9 to about 4.8, about 3.9 to about 4.6, about 3.9 to about 4.4, or any range or subrange thereof. In some preferred embodiments, the personal care composition has a weight ratio of the FeH₃O₃ and/or yellow iron oxide to the Fe₂O₃ and/or red iron oxide of about 2.4 to about 5.1, preferably about 3.4:1 to about 4.2.

The amount of Fe₃O₄ and/or iron (II,III) oxide in the personal care composition may be from about 0.1 to about 1 wt. %, based on the total weight of the personal care composition. For example, the personal care composition may comprise Fe₃O₄ and/or back iron oxide in an amount from about 0.1 to about 1 wt. %, about 0.1 to about 0.8 wt. %, about 0.1 to about 0.6 wt. %, about 0.1 to about 0.4 wt. % about 0.1 to about 0.2 wt. %, about 0.1 to about 0.15 wt. %; from about 0.15 to about 1 wt. %, about 0.15 to about 0.8 wt. %, about 0.15 to about 0.6 wt. %, about 0.15 to about 0.4 wt. % about 0.15 to about 0.2 wt. %; from about 0.2 to about 1 wt. %, about 0.2 to about 0.8 wt. %, about 0.2 to about 0.6 wt. %, about 0.2 to about 0.4 wt. % about 0.2 to about 0.3 wt. %, about 0.2 to about 0.25 wt. %; from about 0.3 to about 1 wt. %, about 0.3 to about 0.8 wt. %, about 0.3 to about 0.6 wt. %, about 0.3 to about 0.4 wt. %; from about 0.4 to about 1 wt. %, about 0.4 to about 0.8 wt. %, about 0.4 to about 0.6 wt. %; from about 0.6 to about 1 wt. %, about 0.6 to about 0.8 wt. %, about 0.8 to about 1 wt. %, or any range or subrange thereof, based on the total weight of the personal care composition.

The amount of FeH₃O₃ and/or yellow iron oxide in the personal care composition may be from about 0.4 to about 5 wt. %, based on the total weight of the personal care composition. For instance, the personal care composition may have FeH₃O₃ and/or yellow iron oxide in an amount from about 0.4 to about 5 wt. %, about 0.4 to about 4 wt. %, about 0.4 to about 3 wt. %, about 0.4 to about 2 wt. %, about 0.4 to about 1.5 wt. %, about 0.4 to about 1 wt. %, about 0.4 to about 0.9 wt. %, about 0.4 to about 0.8 wt. %, about 0.4 to about 0.7 wt. %, about 0.4 to about 0.6 wt. %; from about 0.5 to about 5 wt. %, about 0.5 to about 3 wt. %, about 0.5 to about 1.5 wt. %, about 0.5 to about 1 wt. %, about 0.5 to about 0.9 wt. %, about 0.5 to about 0.8 wt. %, about 0.5 to about 0.7 wt. %; from about 0.6 to about 5 wt. %, about 0.6 to about 3 wt. %, about 0.6 to about 1.5 wt. %, about 0.6 to about 1 wt. %, about 0.6 to about 0.9 wt. %, about 0.6 to about 0.8 wt. %; from about 0.7 to about 5 wt. %, about 0.7 to about 3 wt. %, about 0.7 to about 2 wt. %, about 0.7 to about 1.5 wt. %, about 0.7 to about 1 wt. %, about 0.7 to about 0.9 wt. %; from about 0.8 to about 5 wt. %, about 0.8 to about 3 wt. %, about 0.8 to about 2 wt. %, about 0.8 to about 1.5 wt. %, about 0.8 to about 1 wt. %; from about 1 to about 5 wt. %, about 1 to about 4 wt. %, about 1 to about 3 wt. %, about 1 to about 2 wt. %, about 1 to about 1.5 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

The amount of Fe₂O₃ and/or red iron oxide in the personal care composition may be from about 0.1 to about 2 wt. %, based on the total weight of the personal care composition. In some embodiments, the personal care composition may comprise Fe₂O₃ and/or red iron oxide in an amount of about 0.1 to about 2 wt. %, about 0.1 to about 1.8 wt. %, about 0.1 to about 1.6 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.5 wt. %, about 0.1 to about 0.4 wt. %, about 0.1 to about 0.3 wt. %, about 0.1 to about 0.2 wt. %; from about 0.2 to about 2 wt. %, about 0.2 to about 1.6 wt. %, about 0.2 to about 1.2 wt. %, about 0.2 to about 1 wt. %, about 0.2 to about 0.8 wt. %, about 0.2 to about 0.6 wt. %, about 0.2 to about 0.5 wt. %, about 0.2 to about 0.4 wt. %, about 0.2 to about 0.3 wt. %; from about 0.3 to about 2 wt. %, about 0.3 to about 1.6 wt. %, about 0.3 to about 1.2 wt. %, about 0.3 to about 1 wt. %, about 0.3 to about 0.8 wt. %, about 0.3 to about 0.6 wt. %, about 0.3 to about 0.5 wt. %; from about 0.5 to about 2 wt. %, about 0.5 to about 1.6 wt. %, about 0.5 to about 1.4 wt. %, about 0.5 to about 1.2 wt. %, about 0.5 to about 1 wt. %, about 0.5 to about 0.8 wt. %; from about 0.7 to about 2 wt. %, about 0.7 to about 1.8 wt. %, about 0.7 to about 1.6 wt. %, about 0.7 to about 1.4 wt. %, about 0.7 to about 1.2 wt. %, about 0.7 to about 1 wt. %; from about 1 to about 2 wt. %, about 1 to about 1.8 wt. %, about 1 to about 1.6 wt. %, about 1 to about 1.4 wt. %; from about 1.3 to about 2 wt. %, about 1.3 to about 1.8 wt. %, about 1.3 to about 1.6 wt. %; from about 1.6 to about 2 wt. %, about 1.6 to about 1.8 wt. %, or any range or subrange thereof, based on the total weight of the personal care composition.

The personal care composition may be formulated to have FeH₃O₃ and/or yellow iron oxide in an amount that is greater than the amount of Fe₃O₄ and/or iron (II,III) oxide. Additionally or alternatively, the personal care composition may be formulated to have FeH₃O₃ and/or yellow iron oxide in an amount that is greater than the amount of Fe₂O₃ and/or red iron oxide.

The metal oxide(s) disclosed herein may have a mean particle size of about 5 nm to about 30 μm. In some instances, the mean particle size of the metal oxide may be about 5 nm to about 25 μm, about 10 nm to about 10 μm, or about 15 nm to about 5 μm. In further instances, the metal oxide may a mean particle size of about 5 nm to about 100 nm, about 5 nm to about 75 nm, or about 10 nm to 50 nm. Larger particles sizes may also be useful, such as, e.g., from about 1 μm to about 25 μm, about 5 μm to about 20 μm, or about 10 μm to about 15 μm. The treatment metal oxides may having a coating formed thereon.

Treated metal oxides typically have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, silianes, proteins (collagen or elastin), alkanolamines, silicon oxides, metal oxides, sodium hexametaphosphate, alumina, or glycerol.

In certain embodiments, the mineral UV filter includes one or more zinc oxides, wherein at least one zinc oxide is a treated and/or coated zinc oxide. Examples of treated and/or coated zinc oxides include zinc oxides coated with silica and polymethylhydrogenosiloxane, zinc oxide coated with triethoxycaprylysilane, zinc oxides coated with polymethylhydrogenosiloxane, zinc oxides coated with perfluoroalkyl phosphate and copolymer based on perfluoroalkylethyl as a dispersion in cyclopentasiloxane, and zinc oxides coated with silica and polymethylsilsesquioxane.

In some embodiments, the personal care composition includes an iron oxide system comprising one or more iron oxide that is a treated and/or coated iron oxide. For example, one or more of: FeH₃O₃ and/or yellow iron oxide; Fe₃O₄ and/or iron (II,III) oxide; and/or Fe₂O₃ and/or red iron oxide may be treated and/or coated with at least one of: silica and polymethylhydrogenosiloxane, triethoxycaprylysilane, polymethylhydrogenosiloxane, perfluoroalkyl phosphate and copolymer based on perfluoroalkylethyl as a dispersion in cyclopentasiloxane, and/or silica and polymethylsilsesquioxane. In some embodiments, the personal care composition may comprise FeH₃O₃ (and) Triethoxycaprylylsilane, Fe₂O₃ (and) Triethoxycaprylylsilane, Fe₃O₄ (and) Triethoxycaprylylsilane, or a combination thereof. In at least one embodiment, the personal care composition comprises FeH₃O₃ (and) Triethoxycaprylylsilane, Fe₂O₃ (and) Triethoxycaprylylsilane, and Fe₃O₄ (and) Triethoxycaprylylsilane.

Although the personal care composition may have a reduced amount, be substantially free of, or free of titanium compounds, in some embodiments the personal care composition may include a titanium oxide, including, e.g., a treated and/or coated titanium oxide. Examples of treated and/or coated titanium oxides include those treated with: silica and alumina; alumina and aluminium stearate; iron oxides and iron stearate; silica, alumina and silicone; sodium hexametaphosphate; octyltrimethoxysilane; alumina and stearic acid; alumina and glycerol; alumina and silicone; and combinations thereof. In other embodiments, the personal are composition is free of titanium oxide and/or titanium compounds, regardless of if the titanium oxide and/or titanium compounds are coated and/or treated.

In certain embodiments, the majority of the mineral UV filters in the personal care composition is treated and/or coated. For instance, the personal care composition may have about 50 wt. % or more, about 60 wt. % or more, about 65 wt. % or more, about 70 wt. % or more, about 75 wt. % or more, about 80 wt. % or more, about 90 wt. % or more, about 95 wt. % or more, about 97 wt. % or more, about 98 wt. % or more, about 99 wt. % or more, about 100 wt. %, or any range formed therefrom, relative to the total amount of mineral UV filters, of the mineral UV filters being treated and/or coated. For example, the amount of treated and/or coated mineral UV filter(s) present in the personal care composition may be from about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 5 to about 18 wt. %, about 7 to about 18 wt. %, about 9 to about 15 wt. %, about 11 to about 18 wt. %, about 13 to about 18 wt. %, about 15 to about 18 wt. %; from about 5 to about 15 wt. %, about 7 to about 15 wt. %, about 9 to about 15 wt. %, about 11 to about 15 wt. %, about 13 to about 15 wt. %; from about 5 to about 13 wt. %, about 7 to about 13 wt. %, about 9 to about 13 wt. %, about 11 to about 13 wt. %; from about 5 to about 11 wt. %, about 7 to about 11 wt. %, about 9 to about 11 wt. %; from about 5 to about 9 wt. %, about 7 to about 9 wt. %; from about 5 to about 8 wt. %, about 7 to about 8 wt. %, about 5 to about 7 wt. %, or any range or subrange thereof, based on the total weight of the personal care composition.

In further embodiments, however, the personal care composition may include treated and/or coated metal oxides in an amount from about 0.01 to about 3 wt. %, based on the total weight of the personal care composition. For instance, the amount of treated and/or coated metal oxides in the personal care composition may be from about 0.01 to about 3 wt. %, about 0.01 to about 2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.2 to about 1 wt. %, about 0.2 to about 0.8 wt. %, about 0.2 to about 0.6 wt. %, about 0.2 to about 0.4 wt. %; from about 0.5 to about 1 wt. %, about 0.5 to about 0.8 wt. %, about 0.5 to about 0.6 wt. %; from about 0.8 to about 1 wt. %, about 0.8 to about 1.5 wt. %, about 0.8 to about 2 wt. %, or any range or subrange thereof, based on the total weight of the personal care composition. In certain embodiments, the amount of treated and/or coated metal oxides in the personal care composition is about 0.01 wt. %, about 0.05 wt. %, about 0.1 wt. %, about 0.15 wt. %, about 0.2 wt. %, or any range or subrange thereof, based on the total weight of the personal care composition.

The personal care composition may comprise an organic UV filter, preferably in an effective amount. The amount of organic UV filter may be from about 1 to about 18 wt. %, based on the total weight of the personal care composition. For example, the organic UV filter(s) may be present in the personal care composition in an amount from about 1 to about 18 wt. %, about 1 to about 15 wt. %, about 1 to about 12 wt. %, about 1 to about 9 wt. %, about 1 to about 7 wt. %, about 1 to about 5 wt. %; from about 3 to about 18 wt. %, about 3 to about 15 wt. %, about 3 to about 12 wt. %, about 3 to about 9 wt. %, about 3 to about 7 wt. %; from about 5 to about 18 wt. %, about 5 to about 15 wt. %, about 5 to about 12 wt. %, about 5 to about 9 wt. %; from about 7 to about 18 wt. %, about 7 to about 15 wt. %, about 7 to about 12 wt. %, about 7 to about 9 wt. %; from about 10 to about 18 wt. %, about 10 to about 15 wt. %, about 10 to about 13 wt. %; from about 13 to about 18 wt. %, about 13 to about 16 wt. %, or any range or subrange thereof, based on the total weight of the personal care composition.

The personal care composition may include, or exclude, one or more organic UV filters selected from anthranilic compounds; dibenzoylmethane compounds; cinnamic compounds; salicylic compounds; camphor compounds; benzophenone compounds; y,y-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenyl benzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof. For example, the UV filters may be selected from ethylhexyl methoxycinnamate (octinoxate), oxybenzone, homosalate, octisalate (ethylhexyl salicylate), octocrylene, and a combination of two or more thereof. In at least one embodiment, the personal care composition includes ethylhexyl methoxycinnamate.

The personal care compositions may, in some cases, include one or more SPF enhancer(s). The one or more SPF enhancer(s) may be present in the personal care composition in an amount from about 0.5 to about 10 wt. %, relative to the total weight of the personal care composition. For instance, the personal care composition may comprise the SPF enhancer(s) in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

The one or more SPF enhancer(s) may comprise one or more of butyloctyl salicylate, ethylhexyl methoxycrylene, octyldodecyl neopentanoate, or Pongamia pinnata extract. In certain embodiments, the one or more SPF enhancer(s) are selected from the group consisting of butyloctyl salicylate, ethylhexyl methoxycrylene, octyldodecyl neopentanoate, Pongamia pinnata extract, and a combination of two or more thereof. In some embodiments, the personal care composition comprises at least two, at least three, or at least four of butyloctyl salicylate, ethylhexyl methoxycrylene, octyldodecyl neopentanoate, or Pongamia pinnata extract. For example, the SPF enhancer may comprise butyloctyl salicylate and at least one of ethylhexyl methoxycrylene, octyldodecyl neopentanoate, or Pongamia pinnata extract. In certain embodiments, the SPF enhancer includes butyloctyl salicylate and at least one of ethylhexyl methoxycrylene, or Pongamia pinnata extract.

Additional SPF enhancers that may be included in, or in some cases excluded from, the personal care composition include those selected from photostabilizers and/or antioxidants. Non-limiting examples of photostabilizers include ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, and octocrylene. In some embodiments, the photostabilizer comprises dioctyl butamido triazone, benzylidene malonate polysiloxane, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, octocrylene, or a combination of two or more thereof.

The amount of photostabilizers present in the personal care composition may be from about 0.5 to about 10 wt. %, relative to the total weight of the personal care composition. For instance, the personal care composition may have an amount of photostabilizer(s) ranging from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

The personal care composition may include, or in some cases exclude, one or more SPF enhancer that is an antioxidant. The antioxidant may comprise or be selected from the group consisting of Angelica archangelica, Arnica montana, Camellia sinensis, Cupressus semper, Coffee arabica, Polygonatum multiflorum, Pongamia pinnata extract, Rosmarinus officinalis, Evernia fufuracea, Evernia prunastri, Aventa sativa, vitamin A, vitamin E, peptides, flavones, flavonols, and/or a combination of two or more thereof.

In some embodiments, the antioxidant(s) may be present in an amount from about 0.1 to about 5 wt. %, relative to the total weight of the personal care composition. For instance, the personal care composition may, in some cases, include one or more antioxidant(s) in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, or any range or subrange thereof, based on the total weight of the personal care. The total amount of antioxidant(s) in the personal care composition may in certain embodiments be from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the personal care composition.

The personal care composition may include a cosmetically acceptable carrier other than water. The phrase “cosmetically acceptable” means that the material is compatible with skin and hair. For example, “cosmetically acceptable carrier” means a carrier that is compatible with skin and hair, and is acceptable for application to the body.

The amount of cosmetically acceptable carrier other than water(s) in the personal care composition may be from about 3 to about 35 wt. %, about 5 to about 35 wt. %, about 7 to about 35 wt. %, about 9 to about 35 wt. %, about 11 to about 35 wt. %, about 13 to about 35 wt. %, about 15 to about 35 wt. %, about 18 to about 35 wt. %, about 21 to about 35 wt. %, about 25 to about 35 wt. %, about 30 to about 35 wt. %; from about 3 to about 30 wt. %, about 5 to about 30 wt. %, about 7 to about 30 wt. %, about 9 to about 30 wt. %, about 11 to about 30 wt. %, about 13 to about 30 wt. %, about 15 to about 30 wt. %, about 18 to about 30 wt. %, about 21 to about 30 wt. %, about 25 to about 30 wt. %; from about 3 to about 25 wt. %, about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 3 to about 21 wt. %, about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 3 to about 18 wt. %, about 5 to about 18 wt. %, about 7 to about 18 wt. %, about 9 to about 15 wt. %, about 11 to about 18 wt. %, about 13 to about 18 wt. %, about 15 to about 18 wt. %; from about 3 to about 15 wt. %, about 5 to about 15 wt. %, about 7 to about 15 wt. %, about 9 to about 15 wt. %, about 11 to about 15 wt. %, about 13 to about 15 wt. %; from about 3 to about 13 wt. %, about 5 to about 13 wt. %, about 7 to about 13 wt. %, about 9 to about 13 wt. %, about 11 to about 13 wt. %; from about 3 to about 11 wt. %, about 5 to about 11 wt. %, about 7 to about 11 wt. %, about 9 to about 11 wt. %; from about 3 to about 9 wt. %, about 5 to about 9 wt. %, about 7 to about 9 wt. %; from about 3 to about 8 wt. %, about 5 to about 8 wt. %, about 7 to about 8 wt. %, about 3 to about 6 wt. %, about 4 to about 6 wt. %, or any range or subrange thereof, based on the total weight of the personal care composition.

Non-limiting examples of cosmetically acceptable carriers other than water include glycerin, C₁₋₄ alcohols, organic solvents, fatty alcohols, fatty ethers, fatty esters, polyols, glycols, oils (e.g., vegetable oils, mineral oils, silicone oils, fluoro oils, etc.), laminar lipid materials, or any combinations thereof. As examples of organic solvents, non-limiting mentions can be made of monoalcohols, such as ethyl alcohol, isopropyl alcohol, propyl alcohol, benzyl alcohol, and phenylethyl alcohol.

Non-limiting examples of polyol(s) include those chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, or a combination of two or more thereof. In at least one embodiment, the personal care composition includes one or more polyol(s) comprising panthenol, pantothenic acid, or a combination thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a combination of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The amount of polyol(s) in the personal care composition may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

Additionally or alternatively, the personal care composition may include one or more humectant(s) e.g., in an amount ranging from about 0.01 to about 5 wt. %, based on the total weight of the personal care composition. For example, the one or more humectant(s) may be present in a total amount from about 0.01 to about 4 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 5 wt. %, about 0.8 to about 3 wt. %, about 0.8 to about 2 wt. %, about 0.8 to about 1 wt. %; from about 1.2 to about 5 wt. %, about 1.2 to about 3 wt. %, about 1.2 to about 2 wt. %; from about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Examples of humectants that may be incorporated into the personal care composition include, but are not limited to, polyhydric alcohols such as glycerin, centella asiatica leaf extract, saccharide isomerate butylene glycol, 1,3-propanediol, sorbitol, xylitol or low molecular weight polyethylene glycols (PEGs), polyoxyethylenes or combinations thereof. In some embodiments, the humectant agents are sorbitol (preferably, non-crystal sorbitol), butylene glycol, 1,3-propanediol, glycerin (e.g., vegetable refined glycerin), polyoxyethylene glycol or combinations thereof. In at least one embodiment, the humectants are chosen from glycerin, centella asiatica leaf extract, butylene glycol and 1,3-propanediol. Typically, the humectant is vegetable refined glycerin. Other suitable humectants include, but are not limited to, polyglutamic acid, saccharide isomerate or combinations thereof. In at least one instance, the humectant is saccharide isomerate. Additional examples of humectants include glycerol and its derivatives, urea and its derivatives, sodium pidolate, xylitol, serine, sodium lactate, ectoin and its derivatives, chitosan and its derivatives, collagen, plankton, an extract of Imperata cylindra, beta-glucan, and/or rose hip oil. In certain embodiments, the personal care composition includes a humectant selected from sorbitol, centella asiatica leaf extract, butylene glyco1,1,3-propanediol, glycerin, polyoxyethylene glycol, polyglutamic acid, saccharide isomerate, or a combination of two or more thereof. The personal care composition may include a polyethylene glycol ether, such as PEG-7 trimethylolpropane coconut ether.

The personal care composition may, in some embodiments, include one or more oils, such as those selected from silicone oils, fluoro oils, hydrocarbon-based oils, etc. The amount of oil(s) in the personal care composition may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

The oil used herein may be volatile or non-volatile. The term “volatile oil” relates to an oil that is capable of evaporating on contact with the skin or a keratin fiber in less than one hour, at room temperature and atmospheric pressure. The volatile oil(s) are generally liquid at room temperature and have a non-zero vapor pressure, at room temperature and atmospheric pressure, ranging in particular from 0.13 Pa to 40 000 Pa (10-3 to 300 mmHg). The term “non-volatile oil” relates to an oil that remains on the skin or the keratin fiber, at room temperature and atmospheric pressure, for at least several hours and which in particular has a vapor pressure of less than 10-3 mmHg (0.13 Pa). In some embodiments, the oil may be fatty substance which is in liquid form at room temperature (20-25° C.) and at atmospheric pressure (760 mmHg).

The term “silicone oil” relates to an oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil. Examples of oils, which may be included or excluded from the personal care composition include mineral oils, non-volatile liquid paraffins and/or derivatives thereof, petroleum jelly (petrolatum), polydecenes, hydrogenated polyisobutene such as parleam, squalane, squalene, hydrogenated polyisobutene, hydrogenated polydecene, polybutene, pentahydrosqualene, and combinations thereof.

Non-limiting examples of silicone oils include dimethicone, cyclomethicone, polysilicone-11, phenyl trimethicone, trimethylsilylamodimethicone, and stearoxytrimethylsilane. In some cases, the personal care composition includes dimethicone, and optionally additional oils, including additional silicone oils. Typically, the one or more silicone oil is a non-volatile silicone oil. In some embodiments, the silicone oil is dimethicone, polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. In at least one embodiment, the personal care composition comprises dimethicone having a viscosity of about 1 to about 10 cSt, e.g., about 5 cSt.

The personal care compositions described herein may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the personal care compositions include a synthetic oil selected from the group consisting of polyisobutene, hydrogenated polyisobutene, polydecene, hydrogenated polydecene, and a combination thereof.

The oil may be a non-volatile and hydrocarbon-based, such as hydrocarbon-based oils of plant origin, such as glyceride triesters, which are generally triesters of fatty acids and of glycerol, the fatty acids of which can have varied chain lengths of C₄ to C₂₄, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil. In certain instances, the non-volatile hydrocarbon-based oils are glyceride triesters and in particular to caprylic/capric acid triglycerides, synthetic esters and in particular isononyl isononanoate, oleyl erucate, C₁₂-C₁₅ alkyl benzoate, 2-ethylphenyl benzoate and fatty alcohols, such as octyldodecanol.

The personal care composition may include one or more fatty ester. The fatty ester may be chosen from cetyl ester, ethylhexyl stearate, octyldodecyl neopentanoate, purcellin oil (cetearyl octanoate), isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, and a mixture thereof. Other fatty esters worth mentioning include polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, and polyglyceryl-12 trilaurate. For example, the personal care composition may comprise octyldodecyl neopentanoate, isopropyl palmitate, and ethylhexyl stearate, or a combination of two or more thereof.

The amount of fatty ester(s) in the personal care composition may be from about 1 to about 18 wt. %, about 1 to about 15 wt. %, about 1 to about 12 wt. %, about 1 to about 9 wt. %, about 1 to about 7 wt. %, about 1 to about 5 wt. %; from about 3 to about 18 wt. %, about 3 to about 15 wt. %, about 3 to about 12 wt. %, about 3 to about 9 wt. %, about 3 to about 7 wt. %; from about 5 to about 18 wt. %, about 5 to about 15 wt. %, about 5 to about 12 wt. %, about 5 to about 9 wt. %; from about 7 to about 18 wt. %, about 7 to about 15 wt. %, about 7 to about 12 wt. %, about 7 to about 9 wt. %; from about 10 to about 18 wt. %, about 10 to about 15 wt. %, about 10 to about 13 wt. %; from about 13 to about 18 wt. %, about 13 to about 16 wt. %, or any range or subrange thereof, based on the total weight of the personal care composition.

In some instances, the personal care composition may include one or more fatty alcohol. The fatty alcohols may be selected from those having from about 8 to about 30 carbon atoms, from about 12 to about 22 carbon atoms, and from about 14 to about 22 carbon atoms. These fatty alcohols can be straight or branched chain alcohols and can be saturated or unsaturated. Fatty alcohols that may be include in, or in some cases excluded from, the personal care composition include decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, cis4-t-butylcyclohexanol, myricyl alcohol or a combination thereof. In some cases, the fatty alcohols comprise at least one of or may be chosen from myristyl alcohol, cetyl alcohol, stearyl alcohol, cetearyl alcohol, isostearyl alcohol, oleyl alcohol, isotridecyl alcohol, and a combination thereof.

The amount of fatty alcohol(s) in the personal care composition may be from about 1 to about 18 wt. %, about 1 to about 15 wt. %, about 1 to about 12 wt. %, about 1 to about 9 wt. %, about 1 to about 7 wt. %, about 1 to about 5 wt. %; from about 3 to about 18 wt. %, about 3 to about 15 wt. %, about 3 to about 12 wt. %, about 3 to about 9 wt. %, about 3 to about 7 wt. %; from about 5 to about 18 wt. %, about 5 to about 15 wt. %, about 5 to about 12 wt. %, about 5 to about 9 wt. %; from about 7 to about 18 wt. %, about 7 to about 15 wt. %, about 7 to about 12 wt. %, about 7 to about 9 wt. %; from about 10 to about 18 wt. %, about 10 to about 15 wt. %, about 10 to about 13 wt. %; from about 13 to about 18 wt. %, about 13 to about 16 wt. %, or any range or subrange thereof, based on the total weight of the personal care composition.

The personal care composition typically includes an amount of water. The amount of water present in the personal care composition may be from about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %; about 10 to about 60 wt. %, about 15 to about 60 wt. %, about 20 to about 60 wt. %, about 30 to about 60 wt. %, about 40 to about 60 wt. %, about 50 to about 60 wt. %; about 10 to about 50 wt. %, about 15 to about 50 wt. %, about 20 to about 50 wt. %, about 30 to about 50 wt. %, about 40 to about 50 wt. %, including any ranges and subranges thereof, based on the total weight of the personal care composition.

The personal care composition may optionally include one or more thickening agent(s). The amount of thickening agent in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

The thickener(s) may be chosen from carboxylic acid polymers; crosslinked polyacrylate polymers; polyacrylamide polymers; gums, polysaccharides, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and a combination of two or more thereof.

In certain cases, the personal care composition may be formulated to have thickening agents selected from thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (e.g., carboxymethyl cellulose, methyl cellulose, methylhydroxypropyl cellulose, hydroxyethyl cellulose, hydroxypropyl cellulose, cellulose sulfate sodium salt), and/or starch derivatives (e.g., starch oxide, dialdehyde starch, dextrin, British gum, acetyl starch, starch phosphate, carboxymethyl starch, hydroxyethyl starch, hydroxypropyl starch). In some embodiments, the thickening agent(s) is selected from among hydroxypropyl methylcellulose, polyethylene glycols (PEGs), polyacrylic acids, cross-linked homopolymer of acrylic acid, and acrylates of C₁₀₋₃₀ alkyl acrylate crosspolymer.

The thickening agents may be chosen from xanthan gum, guar gum, biosaccharide gum, cellulose, acacia seneca gum, sclerotium gum, agarose, pechtin, gellan gum, hyaluronic acid, and a combination of two or more thereof. Additionally, the thickening agents may include polymeric thickeners selected from the group consisting of ammonium polyacryloyldimethyl taurate, ammonium acryloyldimethyltaurate/VP copolymer, acrylates copolymers, polyacrylamide, carbomer, acrylates/C₁₀₋₃₀ alkyl acrylate crosspolymer, and a combination of two or more thereof. In at least one preferred embodiment, the personal care composition includes hydroxyethyl acrylate/sodium acryloyldimethyl taurate copolymer.

The personal care compositions may, optionally, comprise one or more amino acid. For example, in certain embodiments, the personal care composition may comprise one or more amino acid. The one or more amino acid may be present in an effective amount. In some embodiments, one or more amino acids are present in an amount from about 0.1 to about 5 wt. %, based on the total weight of the personal care composition. For example, the personal care composition may include one or more amino acids(s) in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, or any range or subrange thereof, based on the total weight of the personal care. The total amount of amino acid(s) in the personal care composition may in certain embodiments be from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the personal care composition.

The one or more amino acids may comprise, or in some cases consist of, arginine, lysine, serine, citrulline, ornithine, creatine, histidine, diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, a salt thereof, and/or a combination of two or more thereof. The personal care composition may include a plurality of amino acids in some instances. For instance, the personal care composition may include a plurality of amino acids selected from taurine, arginine, glycine, lysine, ornithine, proline, and a combination of two or more thereof. In some embodiments, the plurality of amino acids are selected from glycine, arginine, taurine, histidine, tyrosine, tryptophan, a salt thereof, and a combination of two or more thereof. In certain embodiments, the plurality of amino acids comprises taurine and/or a salt thereof, arginine and/or a salt thereof, and glycine and/or a salt thereof.

The personal care composition may be formulated in certain desirable embodiments to have a weight ratio of taurine and/or a salt thereof to arginine and/or a salt thereof to glycine and/or a salt thereof (taurine:arginine:glycine) of (1.5-69):(0.5-40):(0.5-1.5). For instance, the personal care composition may be formulated to have a weight ratio of taurine and/or a salt thereof to arginine and/or a salt thereof to glycine and/or a salt thereof (taurine:arginine:glycine) from about 85:45:1 to about 45:24:1. In some embodiments, the personal care composition has a weight ratio of taurine and/or a salt thereof to arginine and/or a salt thereof to glycine and/or a salt thereof from about 80:42:1 to about 50:30:1; from about 75:40:1 to about 55:29:1; from about 70:37:1 to about 60:31:1, or any range or subrange thereof. In at least one embodiment, the weight ratio of taurine and/or a salt thereof to arginine and/or a salt thereof to glycine and/or a salt thereof (taurine:arginine:glycine) are in a weight ratio of about 65:34:1.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

In certain embodiments, the personal care composition has a pH of from about 4 to about 10.0. In other embodiments, the personal care composition has a pH of from about 4.5 to about 8.0. In some embodiments, the personal care composition has a pH of from about 5 to about 7.5. In other embodiments, the personal care composition has a pH of from about 5.5 to about 7. In further embodiments, the personal care composition has a pH of from about 6 to about 7.

Additional ingredients may be present in the personal care composition. These include ingredients to thicken, preserve, emulsify, add fragrance, adjust the pH, and color. The personal care compositions may include any of the following additional ingredients in an amount of from about 0.01 to about 5 wt. %. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

The personal care composition can comprise additional ingredients, such as proteins (e.g., hydrolyzed vegetable protein, hydrolyzed wheat protein, hydrolyzed milk protein, hydrolyzed silk and hydrolyzed collagen), emollients (e.g., esters, isopropyl myristate, decyl oleate, C₁₂₋₁₅ alkyl benzoate), botanicals (e.g., chamomile, aloe, rosemary), as well as preservatives, dyes, pH adjusters and chelating agents. Additional examples of vitamins that may be included in some personal care compositions include tocopherol, tocopheryl acetate, retinol, and ascorbic acid. The additional ingredients may include clays, such as magnesium silicate and/or magnesium aluminum silicate.

Additionally or alternatively, the personal care composition may comprise an additional ingredient selected from carnosine, cica (asiaticoside), hyaluronic acid and salts thereof (e.g., sodium hyaluronate), magnesium sulfate, saccharide isomerate, a keto-carotenoid (e.g., astaxanthin), nicotinamide adenine dinucleotide (NAD), PEG-7 trimethylolpropane coconut ether, and a combination of two or more thereof. The additional ingredient may include emulsifiers, such as oleth-20, oleth-3 phosphate, diethanolamine, and a combination thereof. The additional ingredient may be selected from anti-aging and/or antioxidants, such as ascorbyl palmitate and Zingiber officinale (ginger) root extract, carnosine, hyaluronic acid.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl) propionaldehyde, ethylvanillin, heliotropin, hexylcinnamaldehyde, amylcinnamaldehyde, 2-methyl-2-(isopropylphenyl) propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes.

The colorants may be present in an amount ranging from about 0.0001 to about 0.4 wt. %, including all percentages and subranges therebetween, based on the total weight of the personal care composition. In some embodiments, the colorants may be present in an amount ranging from about 0.0001 to about 4 wt. %, including all percentages and subranges therebetween, based on the total weight of the personal care composition. In certain embodiments, in order to prevent ingredients from separating, emulsifiers may be added.

In other embodiments, the personal composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); sequestrants, for example, tetrasodium EDTA, and/or their equivalents (0 to 2% by weight); biocides, for example, triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight);

preservatives, for example, phenoxyethanol, formaldehyde solution, parabens, pentanediol or sorbic acid (0 to 2% by weight); pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight); and dyes and pigments that are approved and suitable for cosmetic purposes.

The personal care composition may include one or more vitamins. Examples of vitamins that may be incorporated into the personal care composition include, but are not limited to, vitamin A, vitamin B's such as vitamin B3, vitamin B5, and vitamin B12, vitamin C, vitamin K, and different forms of vitamin E like alpha, beta, gamma or delta tocopherols or their mixtures, and derivatives thereof. In at least one embodiment, the personal care composition comprises niacinamide.

The amount of vitamins(s) in the personal care composition may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

### EXAMPLES

### Example 1

Three non-limiting example personal care compositions (Example Compositions A-C) and three comparative compositions (Comparative Compositions 1-3) were prepared in accordance with aspects of the invention. The formulations for producing Example Compositions A-C and Comparative Compositions 1-3 are shown in Table 1.

TABLE 1

| | Ex. A | Ex. B | Ex. C | Comp. 1 | Comp. 2 | Comp. 3 |
|---|---|---|---|---|---|---|
| US INCI Name | (wt. %) | (wt. %) | (wt. %) | (wt. %) | (wt. %) | (wt. %) |
| Ethylhexyl | 7.5 | 7.5 | 7.5 | 7.5 | 7.5 | 7.5 |
| Methoxycinnamate | | | | | | |
| (Octinoxate) | | | | | | |
| Zinc Oxide and Siliane | 9.4 | 9.4 | 9.4 | 9.4 | 9.4 | 9.4 |
| FeH₃O₃ | 0.622 | 0.529 | 0.715 | 0.448 | 0.574 | 0.338 |
| Fe₂O₃ | 0.17 | 0.17 | 0.17 | 0.082 | 0.292 | 0.172 |
| Fe₃O₄ | 0.139 | 0.139 | 0.139 | 0.123 | 0.239 | 0.14 |
| Butylene Glycol | 1-1.5 | 1-1.5 | 1-1.5 | 1-1.5 | 1-1.5 | 1-1.5 |
| Niacinamide | 4.5-6 | 4.5-6 | 4.5-6 | 4.5-6 | 4.5-6 | 4.5-6 |
| Cyclopentasiloxane | 6.5 | 6.5 | 6.5 | 6.5 | 6.5 | 6.5 |
| Fatty Ester (Octyldodecyl | 4.9 | 4.9 | 4.9 | 4.9 | 4.9 | 4.9 |
| Neopentanoate, Isopropyl | | | | | | |
| Palmitate, and Ethylhexyl | | | | | | |
| Stearate) | | | | | | |
| Polyisobutene | 0.5-1 | 0.5-1 | 0.5-1 | 0.5-1 | 0.5-1 | 0.5-1 |
| Hydroxyethyl | 1.4 | 1.4 | 1.4 | 1.4 | 1.4 | 1.4 |
| Acrylate/Sodium | | | | | | |
| Acryloyldimethyl Taurate | | | | | | |
| Copolymer | | | | | | |
| Preservatives, pH adjusters, | 0.5-1.1 | 0.5-1.1 | 0.5-1.1 | 0.5-1.1 | 0.5-1.1 | 0.5-1.1 |
| emulsifiers, etc. (Oleth- | | | | | | |
| 3 Phosphate, PEG-7 | | | | | | |
| Trimethylolpropane Coconut | | | | | | |
| Ether, Sodium Hyaluronate | | | | | | |
| BHT, Phenoxyethanol, | | | | | | |
| Lactic Acid, Phosphoric | | | | | | |
| Acid, Iodopropynyl | | | | | | |
| Butylcarbamate, and | | | | | | |
| Tocopheryl Acetate) | | | | | | |
| Water | QS to | QS to | QS to | QS to | QS to | QS to |
| | 100 | 100 | 100 | 100 | 100 | 100 |

### Example 2

Example Compositions A-C and Comparative Compositions 1-3, which were each packaged in respective 15 mL airless bottles, were evaluated to assess the level of whitecast on skin. Specifically, a sample from each of Example Compositions A-C and Comparative Compositions 1-3 was applied to the skin on the forearm of panelists having skin categorized as Fitzpatrick 4, 5, or 6. The samples were rubbed on the skin of the respective panelists and then evaluated to determine the amount of whitecast screening on the arm.

Example Compositions A-C each achieved a whitecast value of 100%, which corresponds to being completely transparent with no noticeable white residue. More specifically, a whitecast value of 100% indicates that the percentage of panelists that were able to blend/rub the sunscreen into their forearm without the presence of a whitecast was 100%. Comparative Compositions 1-3 each negatively caused too much whitecast. Specifically, Comparative Compositions 1 and 3 was too light on skin. Comparative Composition 2 was too dark on the skin. Specifically, Comparative Composition 2 altered the appearance of the skin by shading the skin darker, which is undesirable.

### Example 3

Example Compositions A and B, which were contained in a 15 mL airless package, were evaluated to assess the level of whitecast on facial skin of panelists having skin categorized as Fitzpatrick 4, 5, or 6. Specifically, panelists were instructed to take a baseline picture of face before application of the respective composition, then apply one pump (measured to be an average of 0.15 g after 10 pumps, with a range of 0.10 g to 0.18 g) of the respective composition on a finger or directly to face and rub respective composition into skin for blending. Following the blend, a final picture was taken to show a before and after application. Panelists were asked to complete a questionnaire on their experience and observation of how the product blends on their skin.

Example Composition A achieved a whitecast value of 85.7% while Example Composition B achieved a whitecast value of 61.9%. As mentioned in the foregoing Example, the whitecast value represents the percentage of panelists that applied the composition without the presence of a whitecast.

### Example 4

Two non-limiting example personal care compositions (Example Compositions D and E) were prepared in accordance with aspects of the invention. The formulations for producing Example Compositions D and E are shown in Table 2.

TABLE 2

| | Ex. D | Ex. E |
|---|---|---|
| US INCI Name | (wt. %) | (wt. %) |
| Ethylhexyl Methoxycinnamate | 7.5 | 7.5 |
| (Octinoxate) | | |
| Zinc Oxide, and | 9 | 9 |
| Zinc oxide and Siliane | | |
| FeH₃O₃ | 1.09 | 0.856 |
| Fe₂O₃ | 0.30 | 0.234 |
| Fe₃O₄ | 0.24 | 0.19 |
| Petrolatum | 7 | 7 |
| Dimethicone | 1 | 1 |
| Isopropyl Palmitate, Ethylhexyl Stearate, | 6.5 | 6.5 |
| and Octyldodecyl Neopentanoate | | |
| Cetearyl Alcohol | 0.7-1 | 0.7-1 |
| Cetearyl Glucoside | 0.1-0.3 | 0.1-0.3 |
| Polyether-1 | 0.1-0.3 | 0.1-0.3 |
| Polyisobutene | 0.2-0.5 | 0.2-0.5 |
| Hydroxyethyl Acrylate/Sodium | 0.6 | 0.6 |
| Acryloyldimethyl Taurate Copolymer | | |
| Preservatives, pH adjusters, etc. (PEG-7 | 1.5-2 | 1.5-2 |
| Trimethylolpropane Coconut Ether, | | |
| Sodium Hyaluronate, Tocopheryl Acetate, | | |
| Citric Acid, Phosphoric Acid, | | |
| Phenoxyethanol, Oleth-3 Phosphate, | | |
| BHT, Iodopropynyl Butylcarbamate, and | | |
| Butylene Glycol) | | |
| Water | QS to 100 | QS to 100 |

### Example 5

Example Compositions D and E, which were contained in a 15 mL airless package, were evaluated to assess the level of whitecast on skin. Specifically, a sample from each of Example Compositions D and E were applied to the skin on the forearm of the panelists and to the facial skin of the panelists. The procedure for applying the Example Compositions D and E to the panelists' facial skin the same as the procedure described in Example 3. The panelists had skin categorized as Fitzpatrick 4, 5, or 6.

The samples were rubbed onto the skin of the respective panelists and then evaluated to determine the amount of whitecast screening on the arm. Example Compositions D and E both achieved a whitecast screening value of 90.9% on the skin on the forearms of the panelists. Example Composition D achieved a whitecast screening value of 95% on the facial skin of the panelists, while Example Composition E achieved a whitecast screening value of 65% on the facial skin of the panelists.

### Example 6

A non-limiting example personal care compositions (Example Composition F) was prepared in accordance with aspects of the invention. The formulation for producing Example Composition F are shown in Table 3.

TABLE 3

| | Ex. F |
|---|---|
| US INCI Name | (wt. %) |
| Zinc oxide (and) | 14 |
| Triethoxycaprylylsilane | |
| Titanium Dioxide (and) alumina (and) | 5.7 |
| hydrogen dimethicone | |
| FeH₃O₃ (and) Triethoxycaprylylsilane | 2.63 |
| Fe₂O₃ (and) Triethoxycaprylylsilane | 0.68 |
| Fe₃O₄ (and) Triethoxycaprylylsilane | 0.53 |
| Octyldodecyl Neopentanoate | 4 |
| Butyloctyl Salicylate | 5 |
| Polyhydroxystearic Acid | 1 |
| Dimethicone, Cetyl Dimethicone, | 22 |
| Lauryl PEG-9 Polydimethylsiloxyethyl | |
| Dimethicone, and Dimethicone (and) | |
| Dimethicone/PEG-10/15 Crosspolymer | |
| Sodium Chloride | 0.5 |
| Preservatives, pH adjusters, etc. | 1.1-1.5 |
| (Tocopheryl Acetate, Tocopherol, | |
| Phenoxyethanol, Potassium Sorbate, | |
| and Citric Acid) | |
| Anti-aging and/or antioxidant | 0.3-0.9 |
| (Ascorbyl Palmitate and Zingiber | |
| Officinale (Ginger) Root Extract, | |
| Carnosine, Hyaluronic Acid) | |
| Water | QS to 100 |

What is claimed is:

1. A personal care composition comprising:
a mineral UV filter component comprising:
a zinc oxide; and
an iron oxide system comprising:
Fe₃O₄,
FeH₃O₃, and
Fe₂O₃;
wherein the personal care composition has a weight ratio of the FeH₃O₃ to the Fe₃O₄ of about 3.1 to about 6.2 and a weight ratio of the FeH₃O₃ to the Fe₂O₃ of about 2.4 to about 5.1.

2. The personal care composition according to claim 1, wherein the iron oxide system is present in an amount from about 0.6 to about 2.1 wt. %, based on the total weight of the personal care composition.

3. The personal care composition according to claim 1, wherein the iron oxide system is present in an amount from about 0.8 to about 1.25 wt. %, based on the total weight of the personal care composition.

4. The personal care composition according to claim 1, wherein the iron oxide system is present in an amount from about 1.25 to about 2.1 wt. %, based on the total weight of the personal care composition.

5. The personal care composition according to claim 1, wherein the weight ratio of the FeH₃O₃ to the Fe₃O₄ of about 3.2 to about 6.

6. The personal care composition according to claim 1, wherein the weight ratio of the FeH₃O₃ to the Fe₂O₃ of about 2.6 to about 4.9.

7. The personal care composition according to claim 1, wherein the mineral UV filter is present in the amount of about 5 to about 14 wt. %, based on the total weight of the personal care composition.

8. The personal care composition according to claim 1 further comprising:
an organic UV filter selected from ethylhexyl methoxycinnamate, oxybenzone, homosalate, octisalate, octocrylene, and a combination of two or more thereof, wherein the organic UV filter is present in an amount from about 1 to about 18 wt. %, based on the total weight of the personal care composition.

9. The personal care composition according to claim 1 further comprising:
a fatty compound in an amount about 1 to about 10 wt. %, based on the total weight of the personal care composition.

10. The personal care composition according to claim 1 further comprising:
a SPF enhancer comprising butyloctyl salicylate, ethylhexyl methoxycrylene, octyldodecyl neopentanoate, Pongamia pinnata extract, or a combination of two or more thereof.

11. The personal care composition according to claim 1 further comprising:
an amino acid selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

12. The personal care composition according to claim 1, wherein the personal care composition is free of titanium dioxide.

13. The personal care composition according to claim 1, wherein the iron oxide system consists of Fe₃O₄, FeH₃O₃, and Fe₂O₃.

14. The personal care composition according to claim 13, wherein the FeH₃O₃ is present in an amount that is greater than the amount of Fe₃O₄.

15. The personal care composition according to claim 13, wherein the FeH₃O₃ is present in an amount that is greater than the amount of Fe₂O₃.

16. A personal care composition comprising:
a mineral UV filter component comprising:
from about 9 to about 17 wt. % of a zinc oxide; and
an iron oxide system comprising:
Fe₃O₄,
FeH₃O₃, and
Fe₂O₃,
wherein the FeH₃O₃ is present in an amount that is greater than the amount of Fe₃O₄ and the FeH₃O₃ is present in an amount that is greater than the amount of Fe₂O₃, and
wherein all weight percentages are based on the total weight of the personal care composition.

17. The personal care composition according to claim 16 further comprising:
titanium dioxide, wherein the titanium dioxide is present in an amount from about 0.4 to about 8 wt. %.

18. The personal care composition according to claim 16, wherein the personal care composition has a weight ratio of the FeH₃O₃ to the Fe₃O₄ of about 3.1 to about 6.2.

19. The personal care composition according to claim 16, wherein the personal care composition has a weight ratio of the FeH₃O₃ to the Fe₂O₃ of about 2.4 to about 5.1.

20. A personal care composition comprising:
a mineral UV filter component comprising:
a zinc oxide; and
an iron oxide system comprising:
iron (II,III) oxide,
yellow iron oxide, and
red ferric oxide;
wherein the personal care composition has a weight ratio of the yellow iron oxide to the iron (II,III) oxide of about 3.1 to about 6.2 and a weight ratio of the yellow iron oxide to the red ferric oxide of about 2.4 to about 5.1.
